(19) United States
(12) Patent Application Publication
(10) Pub. No.: US 2006/0045904 A1
(43) Pub. Date: Mar. 2, 2006

(54) Joint therapy

(76) Inventor: Barry Aronson, Akron, OH (US)

Correspondence Address: HAHN LOESER & PARKS, LLP, One GOJO Plaza, Suite 300, AKRON, OH, 44311-1076, US

(21) Appl. No.: 11/208,908

(22) Filed: Aug. 22, 2005

Related U.S. Application Data
(60) Provisional application No. 60/603,221, filed on Aug. 20, 2004.

(51) Int. Cl. A61K 38/30; A61F 2/00

(52) U.S. Cl. 424/426; 514/4; 424/423; 514/12

(57) ABSTRACT

A method for synergistically treating a joint of an animal, including introducing a cushioning agent into a joint; and introducing an enzyme or hormone adapted to stimulate cartilage production into the joint. The joint may be subjected to a compression regimen following injection of the cushioning agent and enzyme/hormone. Alternatively, the invention may include a composition for synergistically treating a joint of an animal, comprising a viscous cushioning material adapted to be introduced within a joint space to provide cushioning for the joint, and hormones dispersed through the cushioning material, the hormones operable to increase cartilage production.

### BACKGROUND OF THE INVENTION

Joints in the body enable movement and support weight. Muscular support, cartilage, synovial fluids and other components help joints retain function with use and associated wear and tear. Normal homeostasis in the body keeps all the components in optimized condition but can fail as a result of injury and disease. Reduced joint function and joint pain from a variety of causes is a major concern as a person ages. Active people lose mobility and the enjoyment of many everyday activities. In osteoarthritis and other conditions, the body's own repair mechanisms and homeostasis are overridden by the production of inflammatory agents and insulin-like growth factor binding proteins (IGFBPs).

Orthopedic repair of joints can be a risky major surgical procedure that can leave residual reduced function. In the past, surgical procedures were major with significant risk and treatments were often only minimally effective in advanced cases. Often these repairs require long and painful recuperative periods. While pain and inflammation could at times be controlled with several types of medications, including NSAIDs or steroids, all of these medical treatments had serious side effects, and patients would try them sequentially until the side effects became intolerable and then move on to the next one.

More recently, several new approaches and medications have led to the hope for more permanent more functional improvements. Research has progressed in many areas involving surgical approaches, as well as less invasive techniques to access the joints, the natural history of the articular cartilage, and the response of chondrocytes to both mechanical and chemical means to control chondrocytes proteoglycan synthesis (CPS) and thereby cartilage regrowth. This direction of research obviates the need for cartilage transplants or exogenous growth and reimplantation of the cartilage.

In recent years laparoscopic procedures have introduced minimally invasive repairs. The laparoscopic devices used can provide access to the joint spaces and even facilitate removal of damaged cartilage or smoothing of spurs. Some orthopedic surgeons have also introduced materials to joints through laparoscopes and syringes. Introduction of temporary cushioning agents to act as cartilage replacements, usually composed of some hyaluronic acid-based components, has led to short term relief.

Others have attempted to induce cartilage regeneration through mechanical means or with injection in the joint space with buffered solutions containing any of the insulin like growth factors (IGFs), insulin, similar hormones, formulation of hormones or a combination of these. All hormones and components may be produced from naturally occurring animal sources or non-animal recombinant bacterial sources. Non-animal sources may be preferred to prevent allergy responses. The terms “hormone” and “enzyme” herein both are meant to broadly encompass at least each other.

Additionally or alternatively, low molecular weight molecules can be used that mimic the insulin-like properties needed to act synergistically by preferential binding to IGFBPs to boost the effect of growth hormone in restoring and enhancing chondrocytes proteoglycan synthesis (CPS) and the resultant in vivo cartilage formation. While each of these approaches yields some improvement, they tend to be short term and do not restore the homeostatic balance in the joint.

The ultimate aim of joint repair is to achieve maximal improvement in function and comfort with minimally invasive procedures with rapid return to normal activities and very short periods of down time or debility.

The regrowth of cartilage in vivo in osteoarthritis (OA) has been demonstrated. New substances have been and are being developed to cushion the joint. New substances have been and are being developed that can be used in novel ways to restore the homeostasis in the joint. Studies are now indicating that early compression after intervention yields the best results but may not always be possible after extensive surgery.

### SUMMARY OF THE INVENTION

A new era in the treatment of joint dysfunction will enable earlier intervention with lower risk, improved outcomes and rapid return to normal activities.

Combining and modifying some of the above concepts can lead to a new era in joint repair. The functional outcome can be maximally improved with this invention of materials, procedures and techniques. This invention will also reduce the magnitude of surgery and risk of other orthopedic repairs. Since joint compression also accelerates this process, the normal functioning of the joint is rapidly restored. With these less dramatic interventions and with fewer side effects many conditions can be addressed at a much earlier phase even further improving the results. This invention allows maximum customization to each case based on the nature, location, and severity of the condition. This invention uses various components synergistically to restore the homeostatic balance of the joint thus reversing osteoarthritic changes.

Combined Synergic Therapy (CST) is an invention that heralds a new era of joint care that combines multiple concepts to, in a novel way, cure significant joint damage. This era is marked by minimally invasive procedures able to be used in early phases of joint disease, as well as more advanced states, to regain and maintain normal function by restoring and enhancing homeostasis.

One aspect of the invention involves introducing a cushioning material to the joint. Substances for increasing CPS, such as IGFs and/or low molecular weight molecules that mimic insulin, as well as various forms of insulin, may be added in a way to maximize the development of the articular cartilage. Finally, compression may be introduced into the joint. Rapid resumption of normal function will marshal the innate forces of homeostasis. This customized procedure with the materials used and the devices to apply them constitute CST.

A method to accelerate repair of articular cartilage in joints using the actions of compression, cushioning, and hormones together to maximize cell differentiation to form the chondrocytes that produce cartilage and to stimulate CPS while increasing the joint space to improve function and allow natural use of the joint to compress the forming cartilage and accelerate its development. Existing technology including arthroscopes or syringes to provide access to the joint space, various forms of hyaluronic acid and hormones are combined in a novel method to synergistically achieve short term relief from joint pain and long term improvement in joint function through regrowth of articular cartilage and subsequent remodeling of the joint. Additionally, use of a doped plate in the joint space can be used to keep the hormones in the joint space longer. A novel device to apply this plate is described.

As such, the invention of the present invention may comprise a composition for treating a joint of an animal, including a cushioning agent adapted to be injected into a joint; and an enzyme adapted to stimulate cartilage production. Alternatively, the invention may include a composition for synergistically treating a joint of an animal, comprising a viscous cushioning material adapted to be introduced within a joint space to provide cushioning for the joint, and hormones dispersed through the cushioning material, the hormones operable to increase cartilage production.

In a separate aspect, the invention comprises a method of treating a joint of an animal, comprising the steps of introducing a cushioning agent into the joint space, and introducing an hormone into the joint space, the hormone being operable to stimulate cartilage production, whereby both agents act symbiotically to treat the joint. The method may further include subjecting the joint to a compression regimen.

### DETAILED DESCRIPTION OF THE INVENTION

A preferred embodiment of the present invention comprises a customized combination of enzymes that increase CPS and/or chondrocyte proliferation and/or differentiation. For example, hormones such as IGFs may be utilized. Additionally, cushioning agents such as hyaluronic acid compounds or other agents known in the art can be introduced into the joint space with the hormones. The cushioning agents may be applied in a minimally invasive manner to a joint space and are optionally utilized in conjunction with compression therapy to suppress the chemical products of joint disease and to restore homeostasis in the joint. This allows the joint to better heal itself and repair damage to cartilage. The invention involves a procedure wherein a potential joint space is exposed by traction or inflation by injection of a solution or both. Access to the joint is gained with a syringe, an arthroscope, or a combination of these. Appropriate agents are introduced into the exposed joint space. Compressions of the joint may be introduced as soon as possible and normal function is used to optimize this therapy.

The volume of the joint space may first be evaluated, as well as the condition of the articular cartilage and the other contents of the joint space. In some cases, any needed arthroscopic procedures are performed to clean up the joint space by removing severely degraded cartilage or spurs.

An aspect of the invention is to fill the joint space with the appropriate composition and volume of materials to provide increased or adequate joint function, and optionally to follow this up as rapidly as possible with both physical and occupational therapies to effect movements that cause compression into the joint to maximally elicit a homeostatic response in the joint and to maintain this homeostasis so that the joint resumes normal regrowth and remodeling and subsequent full normal function.

In another aspect of the present invention that would lead to improved joint function, a “plate,” stratum, or layer of material that could be appropriately doped and introduced to the joint space through a syringe or a laparoscope. This plate material can be inert or slowly absorbable. The plate may be designed to be incorporated into the regenerating cartilage. The plate can be formed of solid, gel, or mesh-like material. It may be collagen, fibrin or other organic or even inorganic compounds or other mixed compositions. This plate can also be formed in situ by injecting a doped liquid collagen material. Injections of a shorter acting buffered hormone solution can in various applications enhance the initial action of CST when used together with the plate or in some incipient cases to replace the plate.

The plate, if in the form of a sheet of doped material, can be inserted through a syringe or an arthroscope using an applicator of novel design which is incorporated as an optional part of CST. This plate can be coiled and made of a “memory material” that will uncoil when inserted using a piston driven delivery device or inserted with an applicator that can uncoil the plate as the insertion device is withdrawn. The plate can be of injectable material that will occupy the joint space along with the cushioning material or mix into the matrix of the cushioning material either before, after, or as part of the injection process.

The technology to deliver this plate to the joint space and the physical characteristics of the plate are also novel. The specially prepared plate can be folded or coiled and loaded into an insertion device that uses a piston drive, injector, or other delivery device to position the plate and apply the plate to the desired joint space. This doping plate can be formed of a memory material that will open to the desired configuration when placed into the joint or a material that forms the doped plate in situ.

The composition and administration of the materials is a novel part of one aspect of the present invention, in that it is constituted and administered to enhance and maximize the restoration of homeostasis in the joint. It preferably may contain an adequate volume of filling and cushioning agents to permit increased or enhanced joint function. Customizing the molecular weight and/or the viscosity of, e.g., the hyaluronic acid cushioning agent, for each of the various joints and conditions affecting the joints can be used to optimize this invention for these various applications.

Additionally, enzymes like IGFs may be placed in the needed regions to attract or produce appropriate precursor cells and convert them to chondrocytes, stimulate CPS and cartilage formation in situ, and to inhibit the IGFBPs associated with the original joint damage. This material may be introduced to the joint in the form of a viscous cushioning material along with a plate doped with the needed enzymes in a protracted release form, or with the enzymes bound into the cushioning material in a similar protracted release form. In some applications, normal forms of the enzymes may be used. A protracted release form (PRF) of an enzyme is to be fully released in two to six weeks or longer as indicated for the specific application. PRF can be accomplished by the composition of the enzyme, encapsulation in degradable inert nontoxic materials, or the binding of the enzyme to other components placed into the joint space. This procedure will mimic and enhance the normal compression and release of enzymes that constitute normal homeostasis in the joint.

Another aspect of the invention may also involve compression of the joint post-injection that stimulates cartilage growth. The compression may be accomplished by, e.g., physical or occupational therapy. Additionally, the addition of appropriate hormones or enzymes can boost this cartilage growth. Provision of an appropriate long lasting cushioning agent to the joint can dramatically enhance this process. One aspect of the invention is to combine these products in an optimized fashion.

This CST procedure will yield maximal results with minimal discomfort and rapid resumption of normal joint function by increasing the healing process. This relatively non-destructive process can be repeated as indicated in severe cases until full homeostasis is achieved.

| | |
|---|---|
| CPS | Chondrocytes proteoglycan synthesis |
| CST | Combined Synergistic Therapy |
| IGFBPs | Insulin-like growth factor binding proteins |
| IGFs | Insulin-like growth factors |
| NSAIDs | Nonsteroidal anti-inflammatory drugs |
| OA | Osteoarthritis |
| PRF | Protracted release form of substance (released over a two to |
| | six week period) |

What is claimed is:

1. A composition for treating a joint of an animal, including:
a cushioning agent adapted to be injected into a joint; and,
a hormone adapted to stimulate cartilage production.

2. The composition as recited in claim 1, wherein the cushioning material comprises a hyaluronic acid compound.

3. The composition as recited in claim 1, wherein the hormone is selected from the group consisting of insulin-like growth factors, insulin, and molecules that bind to insulin-like growth factor binding proteins.

4. A method of treating a joint of an animal, comprising the steps of:
introducing a cushioning agent into the joint space; and,
introducing an hormone into the joint space, the hormone being operable to stimulate cartilage production, whereby both agents act synergistically to treat the joint.

5. The method as recited in claim 4, wherein the cushioning agent comprises a hyaluronic acid compound.

6. The method as recited in claim 5, further comprising the step of:
subjecting the joint to a compression regimen after the introduction of the cushioning agent and the hormone, whereby the cushioning agent, the hormone, and the compression regimen act synergistically to treat the joint.

7. The method as recited in claim 6, where in the hormone is an insulin-like growth factor.

8. The method as recited in claim 7, wherein the insulin-like growth factor is contained in a doped plate that is adapted for introduction into the joint.

9. The method as recited in claim 8, wherein the insulin-like growth factor is in a protracted-release form.

10. The method as recited in claim 9, wherein the doped plate is at least partially made of a material that is biologically absorbable.

11. The method as recited in claim 10, wherein the doped plate is at least partially made of collagen or fibrin.

12. The method as recited in claim 4, further comprising the step of:
subjecting the joint to a compression regimen after the introduction of the cushioning agent and the hormone, whereby the cushioning agent, the hormone, and the compression regimen act synergistically to treat the joint.

13. The method as recited in claim 12, wherein the hormone is an insulin-like growth factor.

14. The method as recited in claim 12, wherein the insulin-like growth factor is contained in a doped plate that is adapted for introduction into the joint.

15. The method as recited in claim 14, wherein the insulin-like growth factor is in a protracted-release form.

16. A composition for synergistically treating a joint of an animal, comprising:
a viscous cushioning material adapted to be introduced within a joint space to provide cushioning for the joint, and,
hormones dispersed through the cushioning material, the hormones operable to increase cartilage production.

17. The composition as recited in claim 16, wherein the hormone is selected from the group consisting of insulin-like growth factors, insulin, and molecules that bind insulin-like growth factor binding proteins.

18. The composition as recited in claim 16, wherein the composition is a gel in vivo.

19. The composition as recited in claim 16, wherein the hormone is in a protracted release form.

20. The composition as recited in claim 16, wherein,
the cushioning material is selected from the group consisting of collagen, fibrin, hyaluronic acid compounds, and,
the hormone is selected from the group consisting of: insulin-like growth factors, insulin, and molecules that bind insulin-like growth factor binding proteins.
